(19) United States
(12) Patent Application Publication
(10) Pub. No.: US 2025/0340814 A1
(43) Pub. Date: Nov. 6, 2025

(54) CELL CULTURE APPARATUS AND MEDIUM EXCHANGE METHOD

(71) Applicant: Hitachi High-Tech Corporation, Tokyo (JP)

(72) Inventors: Akane Hongo, Tokyo (JP); Midori Kato, Tokyo (JP); Naohiro Makita, Kyoto (JP); Itsunari Minami, Kyoto (JP)

(73) Assignee: Hitachi High-Tech Corporation, Tokyo (JP)

(21) Appl. No.: 19/268,835

(22) Filed: Jul. 14, 2025

Related U.S. Application Data
(62) Division of application No. 17/604,664, filed on Oct. 18, 2021.

(30) Foreign Application Priority Data
Jul. 2, 2019 (JP) 2019-123315

(51) Int. Cl. C12M 3/06; C12M 1/00; C12N 1/00

(52) U.S. Cl. CPC C12M 27/16; C12M 47/02; C12M 47/10; C12N 1/005; C12N 2513/00; C12N 2525/00

(57) ABSTRACT

By swinging a culture vessel before aspiration removal of a medium, a centrally dense state (83) of the cell population is formed. In the centrally dense state (83), a cell cluster (84) is separated from an outlet port (58a). After aspiration removal of the medium, a new medium is introduced into the culture vessel. After the introduction of the new medium, an overall dispersed state of the cell population is formed.

### TECHNICAL FIELD

The present invention relates to a cell culture apparatus and a medium exchange method, and in particular, to a medium exchange technique.

### BACKGROUND ART

There are a wide variety of cells required in fields such as regenerative medicine and drug discovery, and a culture method suitable for each cell type is required. In particular, in recent years, the development of a three-dimensional cell culture method for culturing a cell mass without adhering it to the bottom surface of the culture vessel has been promoted.

The three-dimensional cell culture method is a method in which a plurality of cells are cultured in a floating state in a medium in a culture vessel without using a scaffold. According to that method, a plurality of cell masses (spheroids) are generated. In carrying out the three-dimensional cell culture method, for example, a culture vessel having a horizontally spread morphology is used. The inner bottom surface of the culture vessel is coated to prevent or reduce cell adhesion, if necessary.

JP-A-2010-268813 (PTL 1) discloses a cell culture apparatus including a mechanism for rotating a culture vessel, a mechanism for discharging a medium from the culture vessel, and a mechanism for injecting a medium into the culture vessel. JP-A-2019-43 (PTL 2) discloses a cell culture apparatus provided with a mechanism for rotating a culture vessel while tilting the culture vessel. In the specification of the present application, in some cases, both a cell existing alone (single cell) and a spheroid composed of a plurality of cells are simply referred to as “cells”.

### CITATION LIST

### Patent Literature

PTL 1: JP-A-2010-268813

PTL 2: JP-A-2019-43

### SUMMARY OF INVENTION

### Technical Problem

Cells used in fields such as regenerative medicine and drug discovery are required to be of the same species and in a uniform state. Therefore, when seeding a plurality of cells on a medium, it is desired to disperse the plurality of cells at a uniform density in the medium in order to make the state of the individual cells uniform. The same applies after the medium is exchanged. On the other hand, when exchanging the medium, it is desired to prevent the excretion of cells and to prevent damage and stress in the cells as much as possible.

An object of the present disclosure is to protect cells during the medium exchange. Alternatively, an object of the present disclosure is to enable a stable culture of a large number of cells.

### Solution to Problem

A cell culture apparatus according to the present disclosure is characterized by including a motion mechanism that holds a culture vessel accommodating a medium containing a plurality of floating cells and causes the culture vessel to exercise a motion and a control unit that manipulates the distribution of the plurality of cells by controlling the motion of the culture vessel and concentrates the plurality of cells around a position away from an outlet port before taking out the medium through the outlet port of the culture vessel, thereby generating a non-uniformly distributed state of the plurality of cells.

A medium exchange method according to the present disclosure is characterized by including a step of concentrating a plurality of floating cells in a medium in a culture vessel while being horizontally separated from an outlet port of the culture vessel, a step of taking out the medium from the culture vessel through the outlet port after concentrating the plurality of cells, a step of introducing a new medium into the culture vessel after the medium is taken out, and a step of totally dispersing the plurality of floating cells in the new medium after the new medium has been introduced.

### Advantageous Effects of Invention

According to the present disclosure, cells can be protected during the medium exchange. Alternatively, according to the present disclosure, a large number of cells can be stably cultured.

### BRIEF DESCRIPTION OF DRAWINGS

FIG. 1 is a conceptual diagram showing a cell culture apparatus according to an embodiment.

FIG. 2 is a front view of a swing mechanism.

FIG. 3 is a schematic front view of a culture vessel.

FIG. 4 is a schematic side view of the culture vessel.

FIG. 5 is a schematic plan view of the culture vessel.

FIG. 6 is a perspective view of the swing mechanism.

FIG. 7 is a diagram showing a connection structure and the operation thereof.

FIG. 8 is a diagram showing an x-axis and a y-axis.

FIG. 9 is a diagram showing a swing motion around the y-axis.

FIG. 10 is a diagram showing a swing motion around the x-axis.

FIG. 11 is a diagram showing an overall dispersed state of cells.

FIG. 12 is a diagram showing a centrally dense state of cells.

FIG. 13 is a diagram showing a state of dense corners of cells.

FIG. 14 is a diagram showing a configuration example of a control unit.

FIG. 15 is a diagram showing a group of parameter tables.

FIG. 16 is a flowchart showing an operation example of the cell culture apparatus.

FIG. 17 is a flowchart showing an operation of forming the overall dispersed state.

FIG. 18 is a flowchart showing an example of an operation at the time of medium exchange.

FIG. 19 is a flowchart showing a first modification of the operation at the time of medium exchange.

FIG. 20 is a diagram showing changes in the cell population during medium aspiration.

FIG. 21 is a flowchart showing a second modification of the operation at the time of medium exchange.

FIG. 22 is a diagram showing a modification of a centrally dense state.

### DESCRIPTION OF EMBODIMENTS

Hereinafter, embodiments will be described with reference to the drawings.

### (1) Outline of the Embodiment

A cell culture apparatus according to the embodiment includes a motion mechanism and a control unit. The motion mechanism holds culture vessels and causes the culture vessels to exercise a motion. The culture vessel is a vessel that accommodates a medium containing a plurality of floating cells. The control unit manipulates the distribution of the plurality of cells in the culture vessel by controlling the motion of the culture vessel, and in particular, concentrates the plurality of cells around a position away from an outlet port before taking out the medium through the outlet port of the culture vessel, thereby generating a non-uniformly distributed state of the plurality of cells.

According to the above configuration, when the medium is taken out from the inside of the cell container through the outlet port, a non-uniformly distributed state including a dense center is formed, and thus, cells can be protected as compared to a state in which a plurality of cells are totally dispersed (i.e., uniformly distributed state). That is, it is possible to prevent floating cells from reaching the outlet port or the vicinity thereof, or it is possible to reduce the possibility thereof. Thereby, the outflow of cells can be avoided or reduced, and the occurrence of damage or stress to the cells can be avoided or reduced.

The outlet port is an opening facing the internal space of the cell container and is provided, for example, at a position close to the inner bottom surface of the cell container. The medium may be aspirated from the outlet port by using the suction force, or the medium may be discharged from the outlet port by using the action of gravity. The non-uniformly distributed state is formed by concentrating a plurality of cells around a position horizontally separated from the outlet port. The concept of horizontally separated positions includes points, lines, or regions. For example, a plurality of cells may be densely packed along the swing axis. The concept of the non-uniformly distributed state can include a mode in which substantially all cells are aggregated in a local region, a mode in which a plurality of cells are distributed so that the density gradually decreases as the distance from the dense center increases, and the like. Even if the peripheral part of the cell population is close to or reaches the outlet port, if the density of the peripheral part is low, a certain degree of protection can be achieved for the entire cell population. Examples of the motion of the culture vessel include a motion, a swing reciprocating motion, a shaking motion, a rotary motion, and the like.

In the embodiment, in a non-uniformly distributed state, a cell cluster horizontally separated from the outlet port is formed. According to the configuration, there is a blank zone between the outlet port and the cell cluster, where no cells are present or only a few cells are present, so that the arrival of cells at the outlet port can be effectively avoided or reduced. When the morphology of the cell cluster changes with the taking out of the medium, the size or density of the cell cluster may be determined in consideration of the change. A cell cluster consists of, for example, 90% or more of cells that are densely packed in a part of a medium that spreads two-dimensionally when viewed from above. In any case, if the center of the cell cluster is far from the outlet port, the cells can be protected as compared with the case where the cells are not densely packed.

The culture vessel according to the embodiment includes an inlet port for introducing a new medium. When viewed from above, the cell cluster is formed between the outlet port and the inlet port. According to the configuration, it is possible to prevent or reduce the occurrence of damage or stress to the cells when the medium is introduced from the inlet port. The outlet port is, for example, an opening provided at the end of the discharge nozzle, and the inlet port is, for example, an opening provided at the end of the introduction nozzle. For example, like the outlet port, the inlet port may be provided at a position close to the bottom surface of the culture vessel.

The culture vessel according to the embodiment has a form extending in both directions of a first axis and a second axis which are orthogonal to each other. The direction of the first axis is parallel to the alignment direction of the outlet port and the inlet port, and the cell cluster extends in the direction of the second axis. If the culture vessel is spread out in a planar manner, changes in individual cell states due to aggregation or depopulation of cells can be avoided, and cells in a constant state can be obtained. Further, according to such a form, a non-uniformly distributed state is likely to be formed by the motion of the culture vessel.

In the embodiment, the first axis and the second axis are virtual axes, respectively, and each is, for example, a swing axis (rotation axis). The first axis and the second axis may be set so as to penetrate the culture vessel, and the first axis and the second axis may be set so as to penetrate the lower side or the upper side of the culture vessel.

In the embodiment, the motion mechanism is a swing mechanism that causes the culture vessel to swing. The control unit controls the swing motion of the culture vessel so that a plurality of cells are densely packed to form a cell cluster. By changing the swing condition, a non-uniformly distributed state and a uniformly distributed state are formed.

In the embodiment, the culture vessel has the swing axis, and the cell cluster is formed by the swing motion of the culture vessel around the swing axis, and the cell cluster is composed of a plurality of cells aggregated in the vicinity of the swing axis. According to the swing motion, the cell cluster can be formed relatively easily. In the embodiment, the swing axis is a virtual axis.

In the embodiment, the control unit has a function of causing an overall dispersed state of a plurality of cells and a function of causing a locally dense state of a plurality of cells as a non-uniformly distributed state. For example, the overall dispersed state is formed at the beginning of the cell culture process, and the locally dense state is formed before the medium exchange. When viewed from above, if a plurality of cells are distributed approximately uniformly across the medium, it can be said to be in the overall dispersed state. The overall dispersed state is a state suitable for cell growth. When viewed from above, if approximately all cells aggregate within a region, resulting in a blank zone in the medium, the state can be said to be a locally dense state.

In an embodiment, the control unit produces the locally dense state before taking out the medium and the overall dispersed state after the introduction of the medium. This configuration adaptively changes the mode of distribution of a plurality of cells according to the situation.

In the embodiment, the culture vessel has a form extending in both directions of a first axis and a second axis which are orthogonal to each other. The cell culture apparatus includes a swing mechanism that executes a first swing motion and a second swing motion. The first swing motion is a motion of causing the culture vessel to perform a swing motion by rotating the culture vessel in the positive and negative directions around the first axis. The second swing motion is a motion of causing the culture vessel to perform a swing motion by rotating the culture vessel in the positive and negative directions around the second axis. The overall dispersed state is formed by causing the culture vessel to perform a swing motion around the first axis and a swing motion around the second axis. The locally dense state is formed by causing the culture vessel to perform the swing motion around the second axis. When forming the locally dense state, the culture vessel may be further subjected to the swing motion around the second axis, if necessary.

In the embodiment, an outlet port is provided on one side of the culture vessel in the direction of the first axis, and the culture vessel further includes an inlet port provided on the other side in the direction of the first axis to introduce a new medium. In the embodiment, the outlet port is provided at one end in the direction of the first axis, and the inlet port is provided at the other end in the direction of the first axis. Each end is a portion near the side wall when viewed from above.

In the embodiment, the control unit controls the motion of the culture vessel so that the locally dense state is repeatedly formed in the process of taking out the medium. For example, in the process of taking out the medium, a locally dense state is repeatedly formed so that the cell cluster does not approach the outlet port below a certain distance. The cell cluster may be imaged so that changes in the morphology thereof can be observed.

The cell culture apparatus according to the embodiment includes a storage unit in which a first parameter set for producing the overall dispersed state and a second parameter set for producing the locally dense state are stored. The control unit produces the overall dispersed state by controlling the motion of the culture vessel according to the first parameter set. In addition, the control unit produces the locally dense state by controlling the motion of the culture vessel according to the second parameter set. The first parameter set and the second parameter set can be obtained in advance by experiments or the like.

The medium exchange method according to the embodiment includes a step of concentrating a plurality of floating cells in the medium in the culture vessel while being horizontally separated from the outlet port of the culture vessel, a step of taking out the medium from the culture vessel through the outlet port after concentrating the plurality of cells, step of introducing a new medium into the culture vessel after the medium is taken out, and a step of totally dispersing the plurality of floating cells in the new medium after the new medium has been introduced.

According to the above configuration, since a plurality of cells are separated from the outlet port in the culture vessel before taking out the medium, the plurality of cells can be protected. After a new medium is introduced, a state in which a plurality of cells are totally dispersed in the culture vessel is formed. It is a state suitable for the growth of a plurality of cells.

In the medium exchange method according to the embodiment, the culture vessel includes an inlet port for introducing a new medium, and a plurality of cells are densely packed, when viewed from above, between the outlet port and the inlet port in the cell container to form a cell cluster. According to this configuration, cells can be protected during both the taking-out of the medium and the introduction of the medium. In the embodiment, the cell cluster is formed at a position horizontally separated from both the outlet port and the inlet port.

In the medium exchange method according to the embodiment, the cell cluster is formed by causing the culture vessel to swing around the swing axis, and the cell cluster has a band-shaped morphology extending along the swing axis. The band-shaped concept may include a rectangle, an ellipse, a bent shape, and the like extending along the swing axis.

### (2) Details of the Embodiment

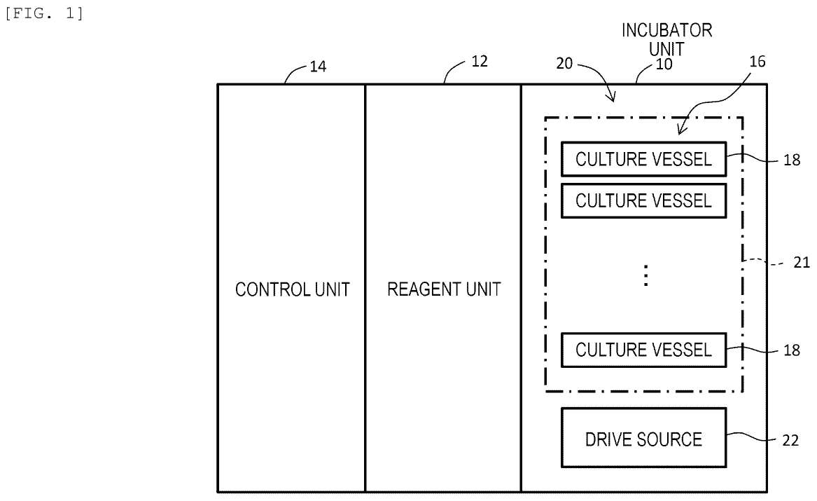

FIG. 1 schematically shows the overall configuration of the cell culture apparatus according to the embodiment. This cell culture apparatus can be used in a three-dimensional cell culture method and is a device capable of automatically introducing a medium, exchanging a medium, seeding cells, and the like. In the embodiment, the cells to be cultured are human cells, for example, population polyfunctional stem cells (iPS cells), nerve cells, and the like. The cells of animals other than humans and the cells of plants may be targeted for culturing. In the three-dimensional cell culture method, a plurality of cells are placed in a floating state in the medium. As a result of culturing, a plurality of spheroids, which are a plurality of cell masses, are formed.

In FIG. 1, the cell culture apparatus is composed of an incubator unit 10, a reagent unit 12, and a control unit 14. The incubator unit 10 includes a swing mechanism 20 as a motion mechanism for causing a culture vessel array 16 to exercise a motion. In the embodiment, the swing mechanism 20 is composed of a holding mechanism 21 that movably holds the culture vessel array 16 and a drive source 22 connected to the holding mechanism 21. The culture vessel array 16 is composed of a plurality of culture vessels 18 aligned in the vertical direction. In the cell culture process, the plurality of culture vessels 18 are each placed in a horizontal posture. As will be described later, the swing mechanism 20 operates when the overall dispersed state and the locally dense state (specifically, the centrally dense state) of the cell population are generated in the individual culture vessels 18.

The reagent unit 12 includes a plurality of medium bottles accommodating new medium, a plurality of pumps for aspirating used medium, a plurality of pumps for feeding new medium, and the like. The control unit 14 controls the operation of each element in the cell culture apparatus. The operation of the drive source 22, in other words, the swing motion of the plurality of culture vessels 18, is controlled by the control unit 14. In the embodiment, the three units 10, 12, and 14 are separated, but the units may be integrated. Alternatively, another unit may be added.

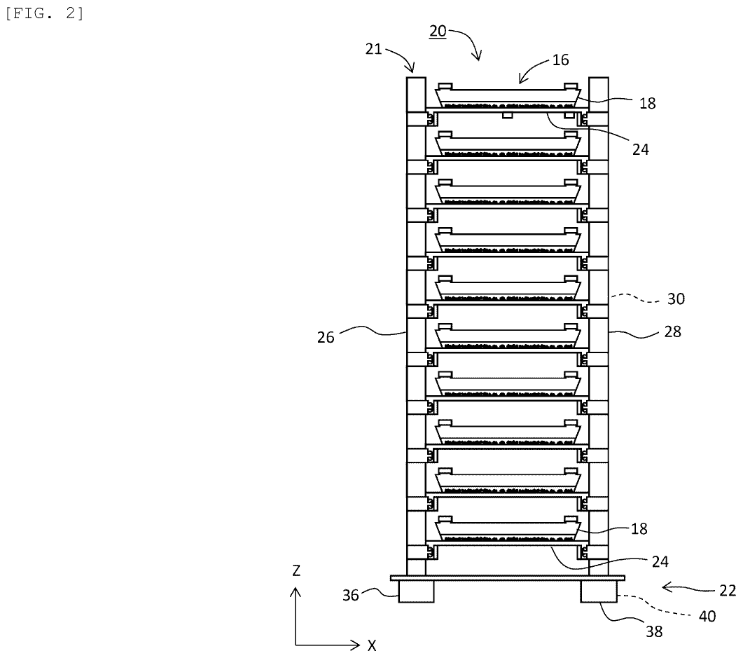

FIG. 2 shows the swing mechanism 20. As described above, the swing mechanism 20 is composed of the holding mechanism 21 and the drive source 22. The holding mechanism 21 includes a plurality of stages 24 and holds the plurality of culture vessels 18 constituting the culture vessel array 16. The holding mechanism 21 includes three movable columns 26, 28, and 30. The three movable columns 26, 28, and 30 are connected to the three corners of each stage 24 with a certain degree of freedom of motion.

The drive source 22 includes three actuators 36, 38, and 40 that apply vertical kinetic forces to the three movable columns 26, 28, and 30. Specifically, the actuator 36 is a mechanism for moving the movable column 26 in the vertical direction, the actuator 38 is a mechanism for moving the movable column 28 in the vertical direction, and the actuator 40 is a mechanism for moving the movable column 30 in the vertical direction. In each drawing, a first horizontal direction is the X direction, a second horizontal direction orthogonal to the X direction is the Y direction, and the direction orthogonal to the X direction and the Y direction is the Z direction.

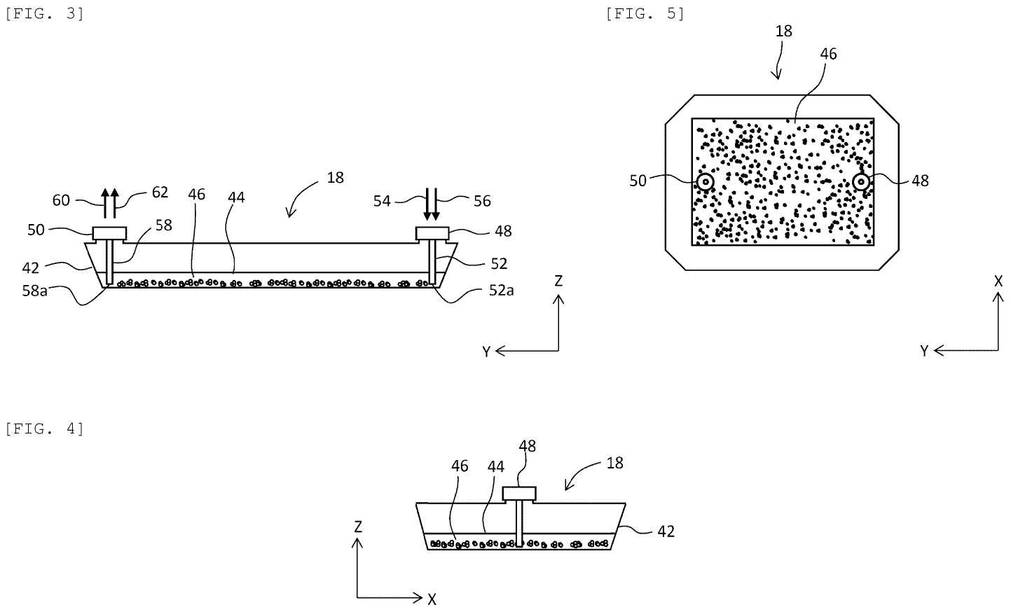

The culture vessel 18 is shown in FIGS. 3 to 5. FIG. 3 is a front view of the culture vessel 18, FIG. 4 is a side view of the culture vessel 18, and FIG. 5 is a top view of the culture vessel 18.

In FIG. 3, the culture vessel 18 includes a vessel body 42 that accommodates a medium 44. The vessel body 42 is made of, for example, a material having chemically stable transparency. Each of the four side walls is inclined. The inner bottom surface of the vessel body 42 is coated, if necessary, to prevent or reduce cell adhesion. The medium 44 contains a plurality of cells 46. An introduction port 48 and a discharge port 50 are provided on the upper part of the vessel body 42.

A nozzle 52 extending downward is connected to the introduction port 48. A lower end opening of the nozzle 52 is an inlet port 52a. The inlet port 52a is close to and faces the inner bottom surface of the vessel body 42. The inlet port 52a is provided in the vessel body 42 in the vicinity of one side end portion in the Y direction, that is, the side surface on one side. The medium 54 sent from the outside and a cell suspension 56 sent from the outside are introduced into the vessel body 42 via the inlet port 52a. The gas required for cell culture is also introduced into the vessel body 42 via the introduction port 48.

A nozzle 58 extending downward is connected to the discharge port 50. A lower end opening of the nozzle 58 is an outlet port 58a. The outlet port 58a is close to and faces the inner bottom surface of the vessel body 42. The outlet port 58a is provided in the vessel body 42 in the vicinity of the other side end portion in the Y direction, that is, the side surface on the other side. The medium is aspirated from the inside of the vessel body 42 through the outlet port 58a, whereby the medium 60 is taken out to the outside. The gas 62 is taken out from the inside of the vessel body 42 through the discharge port 50. By the way, the width of the vessel body 42 in the X direction is in the range of 200 to 250 mm, the width of the vessel body 42 in the Y direction is, for example, in the range of 280 to 320 mm, and the height of the vessel body 42 in the Z direction is, for example, in the range of 20 to 50 mm.

A nozzle extending downward from the bottom surface of the vessel body 42 may be provided, and the medium may be discharged through the nozzle. In that case, the medium may be discharged by the action of gravity, or the medium may be taken out by aspiration. Similarly, as for the nozzle 52, a mode other than the illustrated mode may be adopted. In FIG. 4, the elements already described are designated by the same reference numerals, and the description thereof will be omitted. This also applies to other drawings.

In FIG. 5, when viewed from above, the cell population 46 is two-dimensionally dispersed throughout the culture vessel 18. Microscopically, the cell population 46 is dense but macroscopically, the cell population 46 is distributed at a substantially uniform density. Some cells are present in the vicinity of the inlet port corresponding to the center of the introduction port 48 and the outlet port corresponding to the center of the discharge port 50.

When cell culture is performed, especially when cell seeding is performed, it is necessary to form an overall dispersed state of the cell population as shown in FIG. 5 in order to homogenize the state of individual cells. On the other hand, in the case of discharging the medium, the locally dense state of the cell population, specifically, a centrally dense state of the cell population is formed so that there is no or reduced damage or stress to the cells, and in particular, the excretion of the cells is avoided. The centrally dense state is a state in which a cell cluster is formed while being separated from the inlet port and the outlet port with a gap when viewed from above. The overall dispersed state and the centrally dense state will be described in detail later.

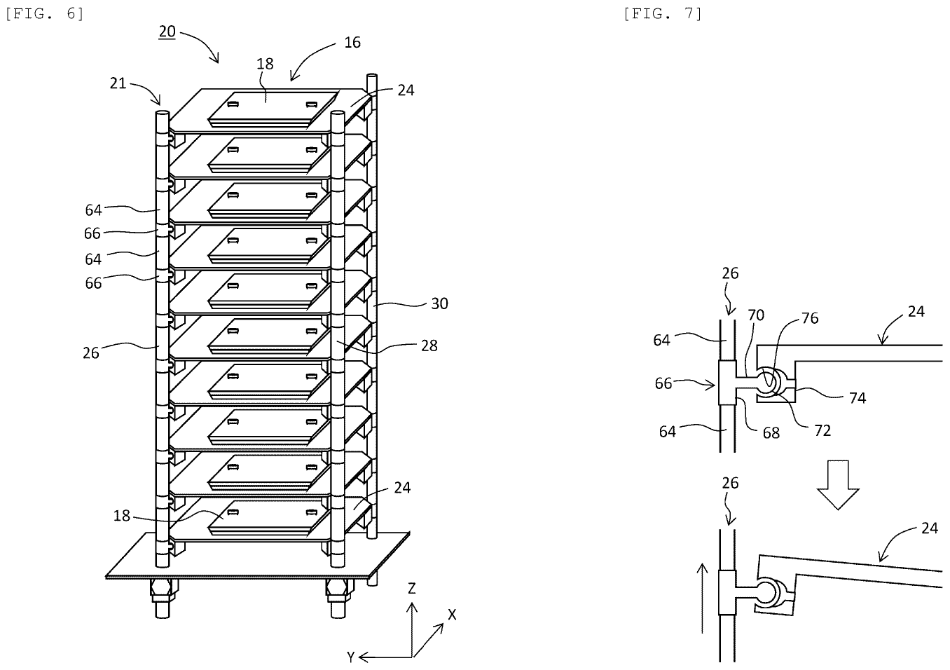

FIG. 6 shows the swing mechanism 20 viewed from an oblique direction. A plurality of culture vessels 18 are held on the plurality of stages 24. Each stage 24 has four corners, in which movable columns 26, 28, and 30 are connected to the three corners. The individual movable columns 26, 28, and 30 have the same configuration, and the configuration will be described below with the movable column 26 as a representative.

The movable column 26 is composed of a plurality of spacers 64 and a plurality of connecting members 66 which are alternately connected. As shown in the upper part of FIG. 7, each connecting member 66 is composed of a tubular member 68 extending in the vertical direction, an arm 70 extending in the horizontal direction from the tubular member 68, and a ball 72 forming an end thereof. On the other hand, a block 74 is provided at the end of the stage 24, and a spherical recess 76 is provided therein. The ball 72 is held by the recess 76. The recess 76 and the ball 72 form a so-called ball joint. Although the stage 24 is held by the connecting member 66, the holding is not fixed, and the motion of the stage 24 is allowed. In the lower part of FIG. 7, the tilting motion of the stage 24 due to the ascending motion of the movable column 26 is illustrated. The configuration shown in FIG. 7 is only an example and other configurations may be adopted.

Referring back to FIG. 6, the posture of each stage 24 can be changed by controlling the vertical positions of the three movable columns 26, 28, and 30, that is, the posture of the culture vessel 18 on each stage can be changed. In the embodiment, the swing mechanism 20 causes each culture vessel 18 to perform the first swing motion and the second swing motion. This will be described in detail below.

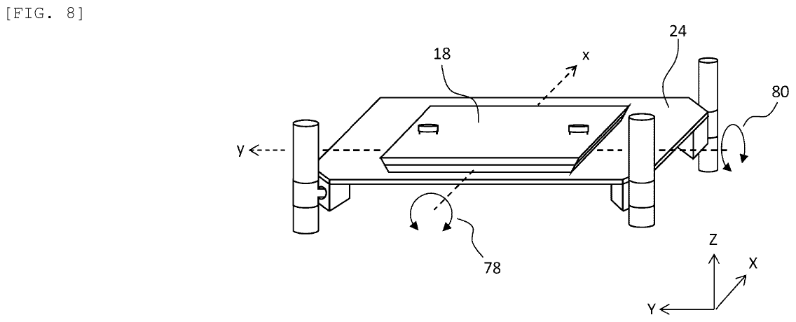

FIG. 8 shows the culture vessel 18 mounted on the stage 24. For the culture vessel 18, the x-axis and the y-axis are defined as virtual swing axes (rotational axes).

The x-axis and the y-axis move with the change in the posture of the culture vessel 18, but when the culture vessel 18 has a horizontal posture, the x-axis is parallel to the X direction and the y-axis is parallel to the Y direction. Further, when viewed from above, the x-axis and the y-axis pass through the center of the culture vessel 18, and both are orthogonal to each other. The swing (rotation) around the x-axis is indicated by reference numeral 78 and the swing (rotation) around the y-axis is indicated by reference numeral 80. The y-axis is parallel to the alignment direction of the inlet port and outlet port. The x-axis corresponds to the central axis of the cell cluster described later.

By controlling the vertical positions of the three movable columns, swings 78 and 80 can be generated. The three movable columns may be set so that the x-axis and y-axis pass below or above the culture vessel 18. In addition, instead of swinging, or together with swinging, reciprocating motion, shaking, rotation, and the like may be adopted.

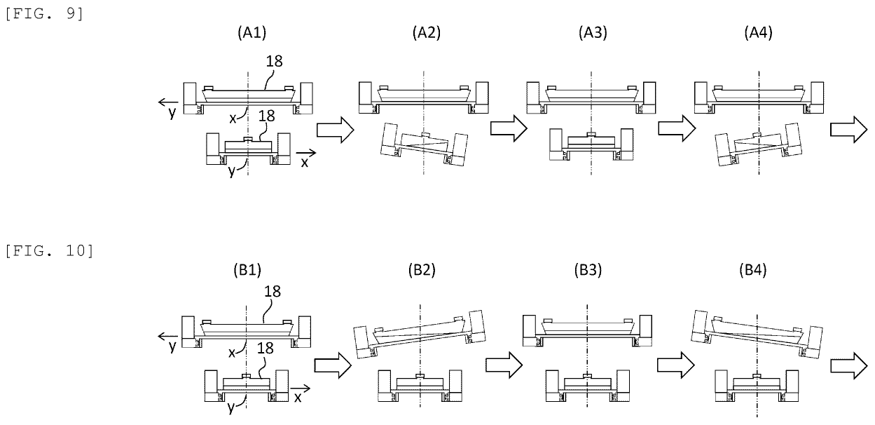

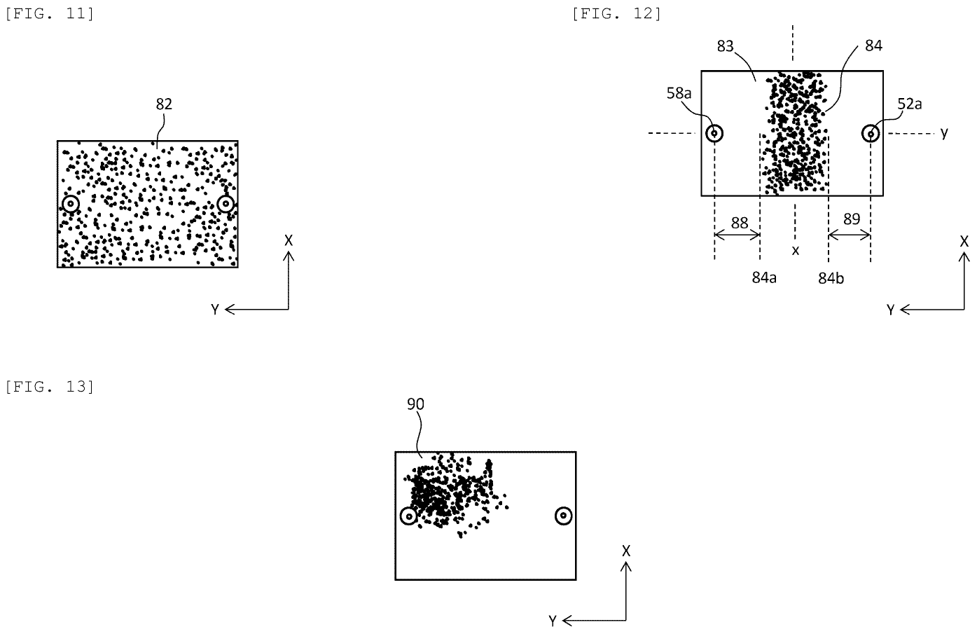

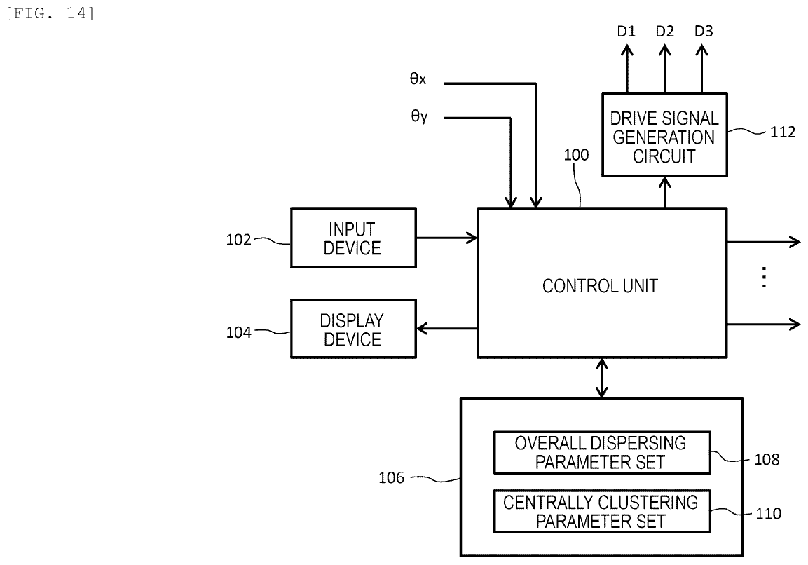

In FIGS. 9 and 10, the upper part of each drawing shows the swing motion around the x-axis, and the lower part of each drawing shows the swing motion around the y-axis. In each drawing, a gradual change in posture is shown from the left side to the right side. Actually, in the embodiment, the swing motions around the two axes are not performed at the same time, and the swing motions around each axis are independently executed. That is, in FIG. 8, the swing motion around the x-axis is not performed and only the swing motion around the y-axis is performed. In FIG. 9, the swing motion around the y-axis is not performed, and only the swing motion around the x-axis is performed.

FIG. 11 shows an overall dispersed state 82 of the cell population. When viewed from above, a plurality of cells are distributed at a substantially uniform density across the culture vessel. The overall dispersed state 82 is formed by causing the culture vessel to perform two swing motions under predetermined conditions. The predetermined conditions are obtained by experiment.

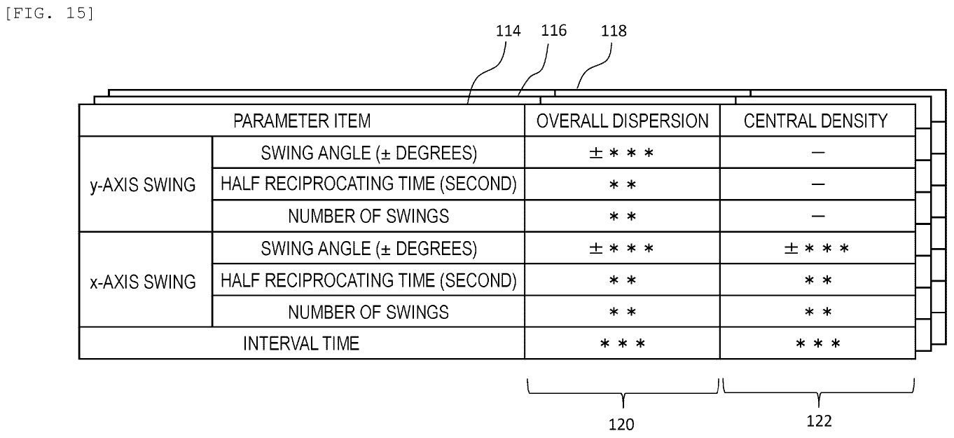

FIG. 12 shows a centrally dense state 83 of the cell population. When viewed from above, the cell populations are aggregated on the x-axis, which is the swing axis, thereby forming a cell cluster 84 having a band-shaped morphology. There is a certain distance 88 between one edge 84a of the cell cluster 84 and the outlet port 58a, and a blank zone is formed there. The certain distance 88 is set so as to prevent the outflow of cells and to prevent the cells from being stressed or damaged more than necessary during the medium discharge process. For example, the certain distance 88 is several centimeters or more, preferably 5 cm or more. The numerical values given in the specification of the present application are merely examples.

There is also a certain distance 89 between the other edge 84b of the cell cluster 84 and the inlet port 52a, and a blank zone is formed there. The certain distance 89 is set so that the cells are not stressed or damaged more than necessary during the medium introduction process. For example, the certain distance 89 is several centimeters or more. For example, the cell cluster 84 is composed of 958, 97%, or 99% or more of cells, with all the cells in the cell container as 100%. However, depending on the situation, the cell cluster 84 may be composed of 90% or more of cells. A small number of cells of 2% or 3% or less may be present in each blank zone.

As the morphology of the cell cluster 84, in addition to a rectangular shape, an elliptical shape, a bent shape, or the like can be considered. In the illustrated example, the cell cluster 84 extends along the x-axis at an intermediate position between the inlet port 52a and the outlet port 58a, but the cell population may be circularly clustered in the center of the x-axis. In any case, it is desirable to control the distribution of the cell population so that the cell population is separated from the inlet port 52a and the outlet port 58a. The centrally dense state 83 is formed under predetermined swing conditions and the predetermined swing conditions are experimentally determined.

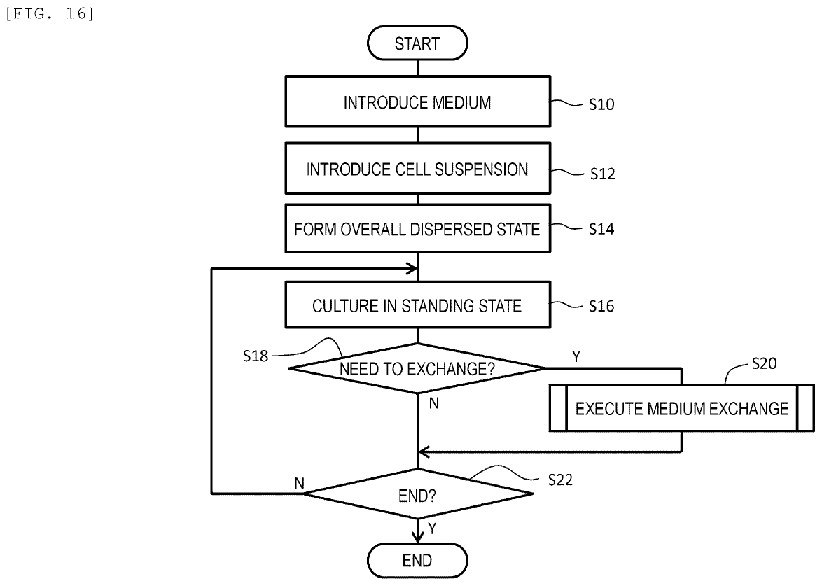

FIG. 13 shows a cell population 90 aggregated on one side (corner portion) in a specific diagonal direction in a culture vessel. Such a distribution state can also be formed by changing the swing conditions. However, in such a distribution state, there are concerns such as cell outflow.

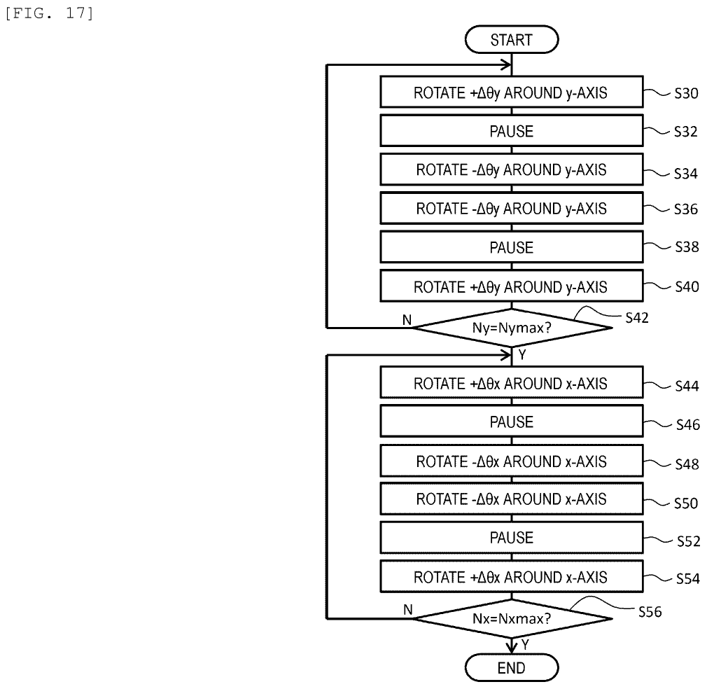

FIG. 14 shows a configuration example of the control unit. A control unit 100 is composed of a processor (for example, a CPU) that executes a program. An input device 102 and a display device 104 are connected to the control unit 100. Also, a memory 106 is connected thereto. An overall dispersing parameter set 108 for forming an overall dispersed state and a centrally clustering parameter set 110 for forming a centrally dense state are stored on the memory 106. Each parameter set 108 and 110 defines a swing condition.

Detection signals from two sensors provided in the swing mechanism are input to the control unit 100. The two detection signals indicate a rotation angle θx around the x-axis and a rotation angle θy around the y-axis. These detection signals are referred to, for example, when feedback-controlling the swing motion around two axes. A drive signal generation circuit 112 is a circuit that generates three drive signals D1, D2, and D3 to be supplied to the three actuators based on the control data from the control unit 100.

The control unit 100 controls the swing motion of the culture vessel according to the centrally clustering parameter set 110 before discharging the medium, thereby causing a centrally dense state of the cell population in the culture vessel field. After that, the medium in the culture vessel is taken out to the outside while maintaining the centrally dense state. Subsequently, a new medium is introduced into the culture vessel. After the introduction of the medium, the control unit 100 controls the swing motion of the culture vessel according to the overall dispersing parameter set 108 to generate an overall dispersed state of the cell population in the culture vessel. The contents of the overall dispersing parameter set 108 and the centrally clustering parameter set 110 may vary depending on the type of culture vessel, the amount of medium, and the like.

FIG. 15 illustrates a plurality of parameter tables 114, 116, and 118. The parameter table to be actually used is selected based on the combination of the type of culture vessel, the amount of medium, and the like. The parameter items include a plurality of parameters (swing angle, half reciprocating time, number of swings) that specify the y-axis swing condition, and a plurality of parameters (swing angle, half reciprocating time, number of swings) that specify the x-axis swing condition, and interval time. The swing angle is an angle in the positive direction or the negative direction, and the half reciprocating time is the time from the horizontal posture to the tilted posture after rotating in the positive or negative direction and then to the horizontal posture again. The interval time is the time to maintain each tilted posture. Reference numeral 120 indicates an overall dispersing parameter set, and reference numeral 122 indicates a centrally clustering parameter set. Actually, those parameter sets 120 and 122 are registered in the memory. The same centrally clustering parameter set may be used regardless of the amount of medium.

For example, as the swing angle around each axis, an angle within the range of 0.1 degrees to 5.0 degrees can be set. For example, as the half reciprocating time, a time within the range of 1.0 seconds to 10.0 seconds can be set. For example, as the number of swings, the number of swings within the range of 1 to 100 times can be set. For example, as the interval time, a time within the range of 0.1 seconds to 10.0 seconds can be set.

When forming an overall dispersed state, for swinging around the y-axis, for example, an angle within the range of 1.0 degree to 3.0 degrees is set as the swing angle, and a time within the range of 1.0 seconds to 3.0 seconds is set as the half reciprocating time, and the number of swings within the range of 2 to 10 is set as the number of swings. Regarding the swing around the x-axis, for example, an angle within the range of 1.0 degrees to 7.0 degrees is set as the swing angle, a time within the range of 1.0 seconds to 3.0 seconds is set as the half reciprocating time, and the number of swings within the range of 2 to 10 is set as the number of swings. Further, as the interval time, a time within the range of 0 seconds to 1.0 seconds is set.

On the other hand, when the centrally dense state is formed, the swing around the y-axis is unnecessary, and only the swing around the x-axis is executed. In that case, for example, an angle within the range of 0.1 degrees to 1.0 degrees is set as the swing angle, and a time within the range of 0.5 seconds to 2.0 seconds is set as the half reciprocating time, and the number of swings within the range of 4 to 50 is set as the number of swings. Further, as the interval time, a time within the range of 0 seconds to 1.0 seconds is set. Of course, individual numbers can change depending on the situation.

Each parameter set may be registered based on the user's input, or the optimum parameter set specified by the experiment may be automatically registered. The swing around the y-axis may be performed when the centrally dense state is formed.

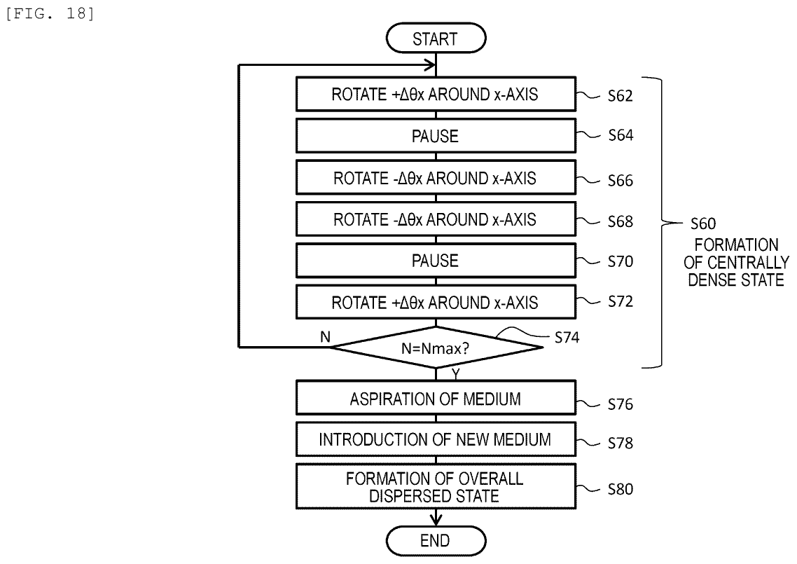

FIG. 16 illustrates the operation of the cell culture apparatus. FIG. 16 shows the content of control by the control unit. In S10, a new medium is introduced into the culture vessel. In S12, a cell suspension is introduced into the culture vessel. In S14, an overall dispersed state is formed by the swing of the culture vessel, specifically, the swing around the y-axis and the subsequent swing around the x-axis. In that case, the swing around the x-axis may be followed by the swing around the y-axis. In S16, cell culture is performed in a state where the culture vessel having a horizontal posture is allowed to stand. For example, when a certain period of time has passed from the introduction of the medium, the necessity of the medium exchange is determined in S18, and the medium exchange is carried out in S20. In S22, it is determined whether or not to end the process, and if it is determined to continue the process, the steps after S16 are executed again.

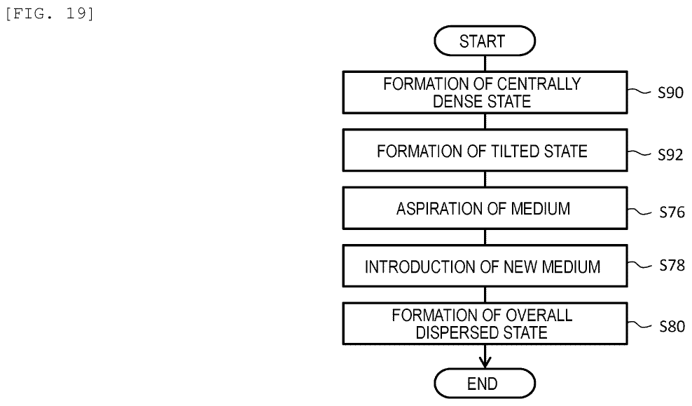

FIG. 17 illustrates the control in forming the overall dispersed state. In the following, the swing angle around the x-axis is referred to as Δθx and the swing angle around the y-axis is referred to as Δθy.

In S30, the control of rotating the culture vessel by +Δθy around the y-axis is executed, and in S32, the control of maintaining the tilted posture of the culture vessel for a certain period of time is executed. In S34, the control of rotating the culture vessel by −Δθy around the y-axis is executed, and subsequently, in S36, the control of rotating the culture vessel by −Δθy around the y-axis is executed. It is also possible to consider S34 and S36 together as a single process. In S38, the control of maintaining the tilted posture of the culture vessel for a certain period of time is executed. In S40, the control of rotating the culture vessel by +Δθy around the y-axis is executed.

In S42, it is determined whether or not the actual number of swings Ny has reached the set value Nymax, and if the number has not reached the set value, step S30 and subsequent steps are executed again. In that case, S40 and S30 can be regarded as a single process. In S42, when it is determined that the actual number of swings Ny has reached the set value Nymax, step S44 and subsequent steps are executed.

In S44, the control of rotating the culture vessel by +Δθx around the x-axis is executed, and in S46, the control of maintaining the tilted posture of the culture vessel for a certain period of time is executed. In

S48, the control of rotating the culture vessel by −Δθx around the x-axis is executed, and subsequently, in S50, the control of rotating the culture vessel by −Δθx around the x-axis is executed. It is also possible to consider S48 and S50 together as a single process. In S52, the control of maintaining the tilted posture of the culture vessel for a certain period of time is executed. In S54, the control of rotating the culture vessel by +Δθx around the x-axis is executed.

In S56, it is determined whether or not the actual number of swings Nx has reached the set value Nxmax, and if the number has not reached the set value, step S44 and subsequent steps are executed again. In that case, S54 and S44 can be regarded as a single process. In S56, when it is determined that the actual number of swings Nx has reached the set value Nxmax, the control ends. In reality, a plurality of culture vessels are processed at the same time.

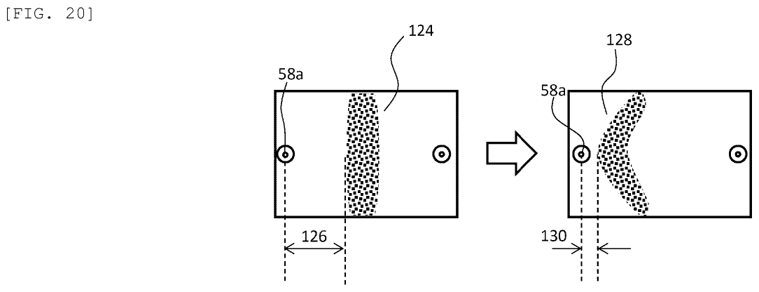

FIG. 18 illustrates the specific contents of S20 in FIG. 16, that is, the control contents at the time of medium exchange. S60 is a step of forming a centrally dense state. Specifically, in S62, the control of rotating the culture vessel by +Δθx around the x-axis is executed, and in S64, the control of maintaining the tilted posture of the culture vessel for a certain period of time is executed. In S66, the control of rotating the culture vessel by −Δθx around the x-axis is executed, and subsequently, in S68, the control of rotating the culture vessel by −Δθy around the x-axis is executed. In S70, the control of maintaining the tilted posture of the culture vessel for a certain period of time is executed. In S72, the control of rotating the culture vessel by +Δθx around the x-axis is executed.

In S74, it is determined whether or not the actual number of swings Nx has reached the set value Nxmax, and if the number has not reached the set value, step S62 and subsequent steps are executed again. In S74, when it is determined that the actual number of swings Nx has reached the set value Nxmax, S76 is executed.

In S76, the used medium is aspirated and removed. In that case, the cell population is densely packed in the middle portion of the culture vessel in the y-axis direction, that is, the cell cluster is separated from the outlet port, and thus, the cells are protected. In S78, a new medium is introduced into the culture vessel. In that case, since the cell cluster is separated from the inlet port, the cells are protected. In S80, an overall dispersed state is formed by swinging the culture vessel. That is, a state suitable for cell growth is formed. Generally, the totally divided state is formed by slowly swinging the culture vessel, and a locally dense state is formed by swinging the culture vessel relatively quickly.

FIG. 19 shows a first modification of the control during the medium exchange. In S90, the centrally dense state of the cell population is formed by swinging the culture vessel. In S92, a state in which the culture vessel is tilted is formed. For example, the posture of the culture vessel is controlled so as to have a tilted posture in which the outlet port is low and the inlet port is high. With such a tilted posture, the aspiration of the medium can be promoted, and the remaining amount of the medium after the aspiration of the medium can be reduced. The posture of the culture vessel may be controlled so as to have a reverse tilted posture in which the outlet port is high and the inlet port is low. In S76, the used medium is removed by aspiration. In S78, a new medium is introduced into the culture vessel. In S80, an overall dispersed state of the cell population is formed by swinging the culture vessel.

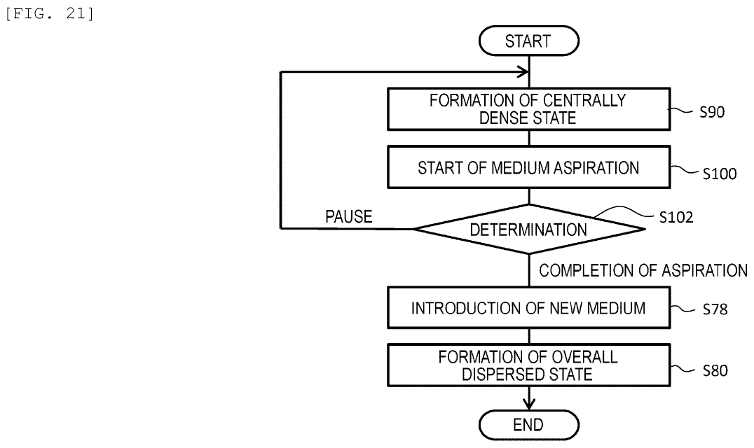

FIG. 20 shows the morphological change of the cell cluster during the medium aspiration process. At the time of formation of the centrally dense state, a cell cluster 124 having a band-like morphology is formed. There is a somewhat large distance 126 between the cell cluster 124 and the outlet port 58a. As the medium aspiration process proceeds, the central portion of the cell cluster approaches the outlet port 58a faster, and the morphology of a cell cluster 128 becomes a bent morphology. At that time, a distance 130 between the cell cluster 128 and the outlet port 58a becomes considerably small. Furthermore, as the medium aspiration process proceeds, some cells may reach the outlet port 58a or the vicinity thereof. In order to avoid such a situation, the centrally dense state may be formed intermittently and repeatedly in the medium aspiration process.

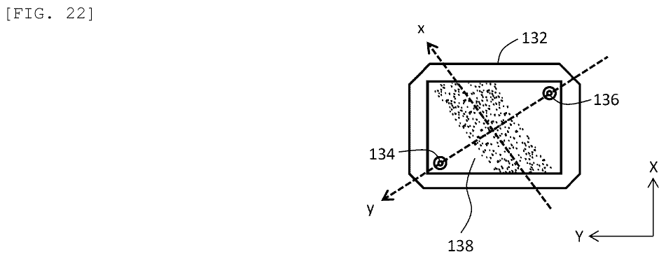

Specifically, a second modification shown in FIG. 21 may be adopted. The same reference numerals are given to the same steps as those already described, and the description thereof will be omitted. In S90, a centrally dense state is formed. In S100, aspiration of the medium is started. In S102, it is determined whether or not to pause the aspiration in a situation where the aspiration is not completed. For example, the pause of aspiration may be determined at regular intervals, or the pause of aspiration may be determined when it is determined that the cell population has approached the outlet port as a result of an analysis of an image of the cell population. In the paused state of aspiration, in S90, the centrally dense state is formed again. When it is determined in S102 that the aspiration is completed, each of step S78 and subsequent steps is executed.

FIG. 22 shows a modification of the centrally dense state. In a culture vessel 132, an outlet port 134 is provided on one side in a specific diagonal direction, and an inlet port 136 is provided on the other side in the diagonal direction. The first swing axis is the y-axis and the second swing axis is the x-axis. By causing the culture vessel 132 to swing around the x-axis, a centrally dense state is formed, that is, a cell cluster 138 is generated. In that state, the used medium is taken out to the outside through the outlet port 134. The cells can be protected even in such a modification.

As described above, according to the above-described embodiment, it is possible to avoid or reduce the occurrence of damage or stress to the cells. In particular, the possibility that cells are excreted can be reduced. If a blank zone is provided between the cell cluster and the outlet port and between the cell cluster and the inlet port, the above-mentioned effect can be obtained more reliably.

1-12. (canceled)

13. A medium exchange method comprising:
a step of concentrating a plurality of floating cells in a medium in a culture vessel while being horizontally separated from an outlet port of the culture vessel;
a step of taking out the medium from the culture vessel through the outlet port after concentrating the plurality of cells;
a step of introducing a new medium into the culture vessel after the medium is taken out; and
a step of totally dispersing the plurality of floating cells in the new medium after the new medium has been introduced.

14. The medium exchange method according to claim 13, wherein
the culture vessel includes an inlet port for introducing the new medium, and
the plurality of cells are densely packed between the outlet port and the inlet port in the culture vessel to form a cell cluster.

15. The medium exchange method according to claim 14, wherein
the cell cluster is formed by swinging the culture vessel around a swing axis, and
the cell cluster has a band-shaped morphology extending along the swing axis.

16. The medium exchange method according to claim 14, wherein:
the culture vessel has a morphology extending in both directions of a first axis and a second axis which are orthogonal to each other,
the direction of the first axis is parallel to the alignment direction of the outlet port and the inlet port, and
the cell cluster extends in the direction of the second axis.

17. The medium exchange method according to claim 14, further comprising:
holding the culture vessel by the motion mechanism, the motion mechanism being a swing mechanism that causes the culture vessel to swing, and
controlling the swing mechanism so that the cell cluster is formed.

18. The medium exchange method according to claim 13, wherein the culture vessel has a morphology in which the culture vessel spreads in both directions of a first axis and a second axis which are orthogonal to each other, and
wherein the method further comprises causing the culture vessel to swing via a swing mechanism, and executing, by the swing mechanism, a first swing motion to rotate the culture vessel in the positive and negative directions around the first axis, and a second swing motion to rotate the culture vessel in the positive and negative directions around the second axis,
wherein performing the swing motion around both the first axis and the swing motion around the second axis causes the plurality of cells to be in an overall dispersed state, and
wherein performing the swing motion around the second axis causes the plurality of cells to be in a locally dense state.

19. The medium exchange method according to claim 13, wherein
the outlet port is provided on one side of the culture vessel in the direction of the first axis, and
the culture vessel further includes an inlet port provided on the other side in the direction of the first axis to introduce the new medium.

20. The medium exchange method according to claim 18, further comprising controlling, by a control unit, the motion of the culture vessel so that the locally dense state is repeatedly formed in the process of taking out the medium.

21. The medium exchange method according to claim 20, further comprising:
storing, in a storage of the control unit, a first parameter set for producing the overall dispersed state and a second parameter set for producing the locally dense state, and
controlling, by the control unit, the motion of the culture vessel according to the first parameter set to produce the overall dispersed state, and controlling, by the control unit, the motion of the culture vessel according to the second parameter set to produce the locally dense state.
